(19) United States
(12) Patent Application Publication
(10) Pub. No.: US 2006/0263445 A1
(43) Pub. Date: Nov. 23, 2006

(54) Enhancement of bacteriacidal activity of silver colloids

(76) Inventor: Steven R. Frank, Niwot, CO (US)

Correspondence Address: WELSH & FLAXMAN LLC, 2000 DUKE STREET, SUITE 100, ALEXANDRIA, VA, 22314, US

(21) Appl. No.: 11/473,329

(22) Filed: Jun. 23, 2006

Related U.S. Application Data
(63) Continuation-in-part of application No. 10/372,795, filed on Feb. 26, 2003.
(60) Provisional application No. 60/693,424, filed on Jun. 24, 2005.

(51) Int. Cl. A61K 33/38; A61K 36/534

(52) U.S. Cl. 424/618; 424/747; 977/906

(57) ABSTRACT

A method for enhancing the antimicrobial effectiveness of silver colloid is provided. The method is achieved by adding to the colloid a compatible agent that modifies the bacterial wall in a manner that increases the uptake of silver by the bacteria. This can be a surfactant or oil that does not cause the silver to compound or precipitate out of suspension.

### CROSS REFERENCE TO RELATED APPLICATIONS

This application is a continuation-in-part of U.S. patent application Ser. No. 10/372,795, filed Feb. 26, 2003, entitled “Treatment of Gastrointestinal Infections”, which is currently pending and which claims the benefit of U.S. Provisional Application Ser. No. 60/693,424, filed Jun. 24, 2005, entitled “Enhancement of Bacteriacidal Activity of Silver Colloids”.

### BACKGROUND OF THE INVENTION

1. Field of the Invention

The invention relates to a method and formulation for the enhancement of bacteriacidal activity of bacterial agents, in particular silver colloid. More specifically, the invention relates to a method and formulation using low concentration additives to increase the biocidal effectiveness of silver colloid.

2. Description of the Prior Art

It has long been known that silver in its pure colloidal form is a powerful antimicrobial. Silver colloids can effectively attenuate many bacteria to varying degrees, with exposures of 20 to 60 minutes. Silver colloids show notable effectiveness when the concentration is greater than 10 ppm.

Since the effectiveness of the silver seems to be tied to its access to the bacteria during the cell division process, increased exposure time is correlated with improved performance up to the point where exposure time is roughly twice the division cycle time. Additionally, increased concentration is strongly correlated with improved bacteriacidal performance. There is an upper limit on the concentration of a pure silver colloid that is set by the amount of silver that can be held in suspension by the weak forces of Brownian motion. This tends to limit the concentration of silver colloids to less than 100 ppm. Exposure time is often limited by the silver compounding with free anions in the environment to produce silver salts offering less antimicrobial activity. Proteins and polysaccharides in the tissue environment also bind with the silver colloid particles rendering them less effective.

Previously, achieving the highest pure concentration silver colloid was all that could be done to ensure optimal antibacterial effectiveness since little could be done to rid the tissue environment of anions, proteins and polysaccharides. Since silver colloids can be produced in pure form up to the estimated limit of colloidal suspension and since the environment determines the useful time of colloid activity, the only way left to improve silver colloid effectiveness is to increase the bacteriacidal activity of the colloid.

Silver colloids are very reactive and there are extremely few compounds or additives that can be combined or even allowed in the presence of the colloid that don't cause it to precipitate or reduce its effectiveness.

As such, a need exists for a method and formulation for achieving a greater bacterial kill with a given concentration of silver colloid.

### SUMMARY OF THE INVENTION

It is, therefore, an object of the present invention to provide a method for enhancing the antimicrobial effectiveness of a silver colloid suspension by adding an agent to the colloid suspension which functions to reduce the surface tension of the peptides found in the bacterial cell walls or otherwise modifying the cell wall and thus rendering them more penetrable to the silver in a colloidal suspension.

The method is achieved by adding specific agents in the form of compounds or oils that enhance the bacteriacidal activity of the silver colloid, while not hindering the aqueous carrier's ability to hold the silver in suspension.

Some examples of additives or agents that offer these capabilities are Polysorbate 20 (also known as Tween 20 or KC-20) and peppermint oil. Both are common food additives.

The advantages of these biocidal enhancers are also effective for silver colloids that are gelled.

Other objects and advantages of the present invention will become apparent from the following detailed description when viewed in conjunction with the accompanying drawings, which set forth certain embodiments of the invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

A method and composition for increasing the bacteriacidal activity of silver colloids has been developed in accordance with the present invention. As will be shown in the following examples, the method is generally achieved by mixing specific compounds and oils in specific concentrations with silver colloid. As those skilled in the art will certainly appreciate, the concentration and type of additive may vary.

Those skilled in the art will understand an aqueous silver colloid to generally consist of silver particles suspended within a liquid medium. In accordance with a preferred embodiment of the present invention, tiny particles of silver are suspended (that is, the dispersed phase), in another phase (that is, the dispersion medium). Also in accordance with a preferred embodiment of the present invention, the dispersion medium is either purified water or an aqueous hydrogel. This gel could be an organic gel such as argarose, xanthan gum, alginates or methylcellulose. The gel could also be synthetic such as carbomers, polyacrylimides or like synthetic gelling agents. These gelling agents and techniques are known in the art.

The silver particles are so small that they remain in suspension indefinitely, unaffected by gravity. Both the silver particles, or dispersed phase, and the dispersion medium may be solid, liquid, or gaseous, although the dispersal of one gas in another is not known as a colloidal dispersion. The particles of a true colloidal dispersion are so small that the incessant bombardment of the molecules of the medium is sufficient to keep the particles in suspension; the random motion of the particles under the influence of this molecular bombardment is called Brownian motion. If, however, the force of gravity is greatly increased in a high-speed centrifuge, the suspension can be broken and the particles made to settle. Silver colloids in accordance with a preferred embodiment of the present invention are produced using an electrolytic process to produce nanometer sized particles (1 to 20 nanometers mostly) in an aqueous colloidal suspension.

The agents that are used to enhance the bacteriacidal effect of the colloid are believed to enhance the performance by reducing the surface tension of the peptides found in the bacterial cell walls and thus rendering them more penetrable to the silver in colloidal suspension. Any substance which renders the cell wall more permeable or vulnerable to the silver would enhance its uptake and improve the bacteriacidal effect.

In accordance with preferred embodiments of the present invention, the silver colloid is mixed with 100 ppm peppermint oil and/or 0.2% by volume polysorbate 20 (Tween 20), for example 2 ml/liter, resulting in various compositions including silver colloid, peppermint oil and/or polysorbate 20. It is also contemplated that the percentages of each ingredients: silver colloid, peppermint oil and polysorbate 20 could be varied to create different compositions in accordance with the present invention. As those skilled in the art will certainly appreciate, various compositions of surfactant and protein modifying ingredients equivalent to Polysorbate 20 may be chosen to achieve these goals without departing from the spirit of the present invention.

Laboratory testing has demonstrated that the addition of the peppermint oil to the silver colloid produces a synergistic effect. The synergistic effect results from the fact that the peppermint oil does not bind with, nor render inactive, the colloid, but rather enhances the anti-microbial activity by a couple orders of magnitude.

The following examples demonstrate the efficacy of the present treatment method and composition, while also disclosing specific embodiments of the present invention:

### EXAMPLE 1

### Results Presented in Graph 1

Silver Colloid vs Silver Colloid+Peppermint Oil in Treating E. Coli, Staph and Strep

E. coli

Methods:

100 μl of an unknown concentration of bacteria was added to 5 ml of silver colloid and silver colloid with 100 ppm peppermint oil. All solutions contained approximately 50 ppm of silver. All solutions were incubated at 37° C. before the addition of bacteria.

The bacteria in the silver colloid solutions was incubated at 37° C. for 30 minutes. After the given amount of time 100 μl of the bacteria-silver colloid solution was plated on LB agar plates and incubated overnight at 37° C. The following day the colonies were counted.

Control Plates:

The original culture was diluted by serial dilution. 100 μl of bacteria was added to 900 μl of Deionized (DI) water, and 100 μl of the new DI water-bacteria solution was added to another 900 μl of DI water. This was repeated a total of 6 times to obtain a −5 and −6 dilution. 100 μl of these dilutions were then plated on LB-agar plates and grown over night at 37° C.

Results:

Control Plates:

Dilutions of −5 and −6 were plated and grown over night at 37° C. The colonies were counted and the quantity of bacteria in the original culture was determined.

| | | CFU in 100 μl | | CFU/ml in |
|---|---|---|---|---|
| | | of original | | original |
| Dilution | Colonies | culture | Average | culture |
| −5 | 123 | 1.23 × 10⁷ | | |
| −6 | 16 | 1.6 × 10⁷ | 1.415 × 10⁷ | 1.415 × 10⁸ |

Calculating the Number of Colonies Plated:

100 μl of bacteria was added to 5 ml of silver colloid solution

1.415×10⁷ CFU/5000 μl=2.83×10³ CFU/μl in the silver colloid solution

100 μl of the silver colloid solution with bacteria was plated.

100 μl×2.83×10³ CFU/μl=2.83×10⁵ CFU

The total number of colonies plated was 2.83×10⁵

After 30 minute:

| Solution | $\frac{Colonies Remaining}{Total Colonies}$ | Log Kill Ratio |
|---|---|---|
| Silver colloid | $\frac{158}{2.83 \times {10}^{5}}$ | 3.25 |
| Silver colloid +peppermint oil | $\frac{0}{2.83 \times {10}^{5}}$ | 5.45 |

Log Kill Calculation:

$$( \frac{Total \# of Colonies}{Surviving Colonies} ) \times {Log}_{10}$$

Log10 of (Total colonies plated/surviving colonies)

or Log10(2.83×10⁵/158)=3.25 and Log10(2.83×10⁵/1)=5.45. We replace the zero with a one and accept that the actual results would have been higher if we had sufficient inoculum to ensure some survivors.

Staph

Methods:

5 ml of 50 ppm silver colloid with and without 100 ppm peppermint oil were used. The solutions were incubated at 37° C. before addition of bacteria. After the solutions were at 37° C., 100 μl of bacteria was added to tubes containing 5 mls of silver colloid and stomach rescue. The solutions were incubated at 37° C. for 30 minutes. 100 μl of the solution was then plated on LB-agar plates and grown for 40 hours at 37° C.

Control Plates:

The original culture was diluted by serial dilution. 100 μl of bacteria was added to 900 μl of DI water, and 100 μl of the new DI water-bacteria solution was added to another 900 μl of DI water. This was repeated a total of 6 times to obtain a −6. 100 μl of the −5, and −6 dilutions were then plated on LB-agar plates and grown over night at 37° C.

Results:

Control Plates

| | | CFU in 100 μl | | CFU/ml in |
|---|---|---|---|---|
| | | of original | | original |
| Dilution | Colonies | culture | Average | culture |
| −5 | 80 | 8.0 × 10⁶ | | |
| −6 | 8 | 8.0 × 10⁶ | 8.0 × 10⁶ | 8.0 × 10⁷ |

With 8.0×10⁶ CFU placed in 5 ml, in 100 μl taken from the 5 ml there would be about 1.6×10⁵ CFU plated if no CFU were killed.

Calculating the Number of Colonies Plated:

100 μl of bacteria was added to 5 ml of silver colloid solution

8.0×10⁶ CFU/5000 μl=1.6×10³ CFU/μl in the silver colloid solution

100 μl of the silver colloid solution with bacteria was plated.

100 μl×1.6×10³ CFU/μl=1.6×10⁵ CFU

The total number of colonies plated was 1.6×10⁵

Experimental Plates

After 30 Minutes:

| Solution | $\frac{Colonies Remaining}{Total Colonies}$ | Log Kill Ratio |
|---|---|---|
| Silver colloid | $\frac{248}{1.6 \times {10}^{5}}$ | 2.8 |
| Silver colloid +Peppermint oil | $\frac{26}{1.6 \times {10}^{5}}$ | 3.8 |

Log Kill Calculation:

$$( \frac{Total \# of Colonies}{Surviving Colonies} ) \times {Log}_{10}$$

Step

Methods:

5 ml of the silver colloid with 100 ppm of peppermint oil, and silver colloid were incubated at 37° C. before addition of bacteria. Both solutions contained 50 ppm of silver. After the solutions were at 37° C., 100 μl of bacteria was added to each 5 ml solution. The solutions were incubated at 37° C. for 40 minutes. 100 μl of the solution was then plated on LB-agar plates and incubated for 40 hours at 37° C.

Control Plates:

The original culture was diluted by serial dilution. 100 μl of bacteria was added to 900 μl of DI water, and 100 μl of the new DI water-bacteria solution was added to another 900 μl of DI water. This was repeated a total of 6 times to obtain a −6. 100 μl of the −5, and −6 dilutions were then plated on LB-agar plates and grown over night at 37° C.

Results:

Control Plates:

| | | CFU in 100 μl | | CFU/ml in |
|---|---|---|---|---|
| | | of original | | original |
| Dilution | Colonies | culture | Average | culture |
| −5 | 40 | 4.0 × 10⁶ | | |
| −6 | 5 | 5.0 × 10⁶ | 4.5 × 10⁶ | 4.5 × 10⁶ |

Approximately 4.5×10⁶ CFU were added to each 5 ml solution. 100 μl was then plated after incubation. About 9.0×10⁴ CFU would be plated if no CFU were killed during the incubation.

Calculating the Number of Colonies Plated:

100 μl of bacteria was added to 5 ml of silver colloid solution

4.5×10⁶ CFU/5000 μl=9×10² CFU/μl in the silver colloid solution

100 μl of the silver colloid solution with bacteria was plated.

100 μl×9×10² CFU/μl=9×10⁴ CFU

The total number of colonies plated was 9×10⁴

After 40 Minutes:

| | Colonies | Log Kill |
|---|---|---|
| Solution | counted | Ratio |
| Silver | 2000 | 1.65 |
| Colloid | | |
| Silver | 0 | 5 |
| colloid + | | |
| peppermint | | |
| oil | | |

Log Kill Calculation:

$$( \frac{Total \# of Colonies}{Surviving Colonies} ) \times {Log}_{10}$$

### EXAMPLE 2

### Results Presented in Graph 2

Silver Colloid vs Silver Colloid+KC-20 in Treating E. Coli, Staph and Strep

E. coli

Methods:

500 μl of an unknown concentration of bacteria was added to 5 ml of silver colloid, and silver colloid with 1% KC-20. Both solutions contained 50 ppm of silver. All solutions were incubated at 37° C. before the addition of bacteria.

The bacteria in the silver colloid solutions incubated at 37° C. for 60 minutes. After the given amount of time 100 μl of the bacteria-silver colloid solution was plated on an LB agar plate and incubated overnight at 37° C. The following day the colonies were counted.

Control Plates:

The original culture was diluted by serial dilution. 100 μl of bacteria was added to 900 μl of DI water, and 100 μl of the new DI water-bacteria solution was added to another 900 μl of DI water. This was repeated a total of 6 times to obtain a −5 and −6 dilution. 100 μl of these dilutions were then plated on LB-agar plates and grown over night at 37° C.

Results:

Control Plates:

Dilutions of −5 and −6 were plated and grown over night at 37° C. The colonies were counted and the quantity of bacteria in the original culture was determined.

| | | CFU in 100 μl of | | CFU/ml in |
|---|---|---|---|---|
| Dilution | Colonies | original culture | Average | original culture |
| −5 | 200 | 2.00 × 10⁷ | | |
| −6 | 19 | 1.90 × 10⁷ | 1.95 × 10⁷ | 1.95 × 10⁸ |

Calculating the Number of Colonies Plated:

500 μl of bacteria was added to 5 ml of silver solution bringing the total volume to 5.5 ml.

9.75×10⁷ CFU/5500 μl=1.77×10⁴ CFU/μl in the silver solution 100 μl of the silver solution with bacteria was plated.

100 μl×1.77×10⁴ CFU/μl=1.77×10⁶ CFU

If no colonies were killed during the incubation in silver colloid there should be approximately 1.77×10⁶ CFU on each plate.

After 60 Minutes:

| Solution | $\frac{Colonies Remaining}{Total Colonies}$ | Log Kill Ratio |
|---|---|---|
| Silver colloid | $\frac{2000}{1.77 \times {10}^{6}}$ | 2.95 |
| Silver colloid +1% KC-20 | $\frac{215}{1.77 \times {10}^{6}}$ | 3.92 |

Log Kill Calculation:

$$( \frac{Total \# of Colonies}{Surviving Colonies} ) \times {Log}_{10}$$

Staph

Methods:

A solution of silver colloid with 1% KC-20 was made by adding 50 μl of KC-20 to 5 ml of silver colloid. Silver colloid solutions contained 50 ppm of silver. The solutions were incubated at 37° C. before addition of bacteria. After the solutions were at 37° C., 100 μl of bacteria was added to tubes containing 5 mls of silver colloid-KC-20, or silver colloid. The solutions were incubated at 37° C. for 30 minutes. 100 μl of the solution was then plated on LB-agar plates and grown for 40 hours at 37° C.

Control Plates:

The original culture was diluted by serial dilution. 100 μl of bacteria was added to 900 μl of DI water, and 100 μl of the new DI water-bacteria solution was added to another 900 μl of DI water. This was repeated a total of6 times to obtain a −6. 100 μl of the −5, and −6 dilutions were then plated on LB-agar plates and grown over night at 37° C.

Results:

Control Plates

| | | | | CFU/ml in |
|---|---|---|---|---|
| | | CFU in 100 μl of | | original |
| Dilution | Colonies | original culture | Average | culture |
| −5 | 80 | 8.0 × 10⁶ | | |
| −6 | 8 | 8.0 × 10⁶ | 8.0 × 10⁶ | 8.0 × 10⁷ |

With 8.0×10⁶ CFU placed in 5 ml, in 100 μl taken from the 5 ml there would be about 1.6×10⁵ CFU plated if no CFU were killed.

Calculating the Number of Colonies Plated:

100 μl of bacteria was added to 5 ml of silver solution

8.0×10⁶ CFU/5000 μl=1.6×10³ CFU/μl in the silver solution

100 μl of the silver solution with bacteria was plated.

100 μl×1.6×10³ CFU/μl=1.6×10⁵ CFU

If no colonies were killed during the incubation in silver colloid there should be approximately 1.6×10⁵ CFU on each plate.

After 30 Minutes:

| Solution | $\frac{Colonies Remaining}{Total Colonies}$ | Log Kill Ratio |
|---|---|---|
| Silver colloid | $\frac{248}{1.6 \times {10}^{5}}$ | 2.8 |
| Silver Colloid +1% KC-20 | $\frac{0}{1.6 \times {10}^{5}}$ | 5.2 |

Log Kill Calculation:

$$( \frac{Total \# of Colonies}{Surviving Colonies} ) \times {Log}_{10}$$

Strep

Methods:

5 ml of silver colloid, and silver colloid with 1% KC-20 were incubated at 37° C. before addition of bacteria. After the solutions were at 37° C., 100 μl of bacteria was added to each 5 ml solution. The solutions were incubated at 37° C. for 40 minutes. 100 μl of the solution was then plated on LB-agar plates and incubated for 40 hours at 37° C.

Control Plates:

The original culture was diluted by serial dilution. 100 μl of bacteria was added to 900 μl of DI water, and 100 μl of the new DI water-bacteria solution was added to another 900 μl of DI water. This was repeated a total of 6 times to obtain a −6. 100 μl of the −5, and −6 dilutions were then plated on LB-agar plates and grown over night at 37° C.

Results:

Control Plates:

| | | | | CFU/ml in |
|---|---|---|---|---|
| | | CFU in 100 μl of | | original |
| Dilution | Colonies | original culture | Average | culture |
| −5 | 1000 | 1.0 × 10⁸ | | |
| −6 | 120 | 1.2 × 10⁸ | 1.1 × 10⁸ | 1.1 × 10⁹ |

Approximately 1.1×10⁸ CFU were added to each 5 ml solution. 100 μl was then plated after incubation. About 2.2×10⁶ CFU would be plated if no CFU were killed during the incubation.

Calculating the Number of Colonies Plated:

100 μl of bacteria was added to 5 ml of silver solution

1.10×10⁸ CFU/5000 μl=2.2×10⁴ CFU/μl in the silver solution

100 μl of the silver solution with bacteria was plated.

100 μl×2.2×10⁴ CFU/μl=2.2×10⁶ CFU

The total number of colonies plated was 2.2×10⁶

After 40 Minutes:

| | Colonies | |
|---|---|---|
| Solution | counted | Log Kill Ratio |
| Silver Colloid | 1500 | 3.2 |
| Silver Colloid + | 150 | 4.1 |
| 1% KC-20 | | |

Log Kill Calculation:

$$( \frac{Total \# of Colonies}{Surviving Colonies} ) \times {Log}_{10}$$

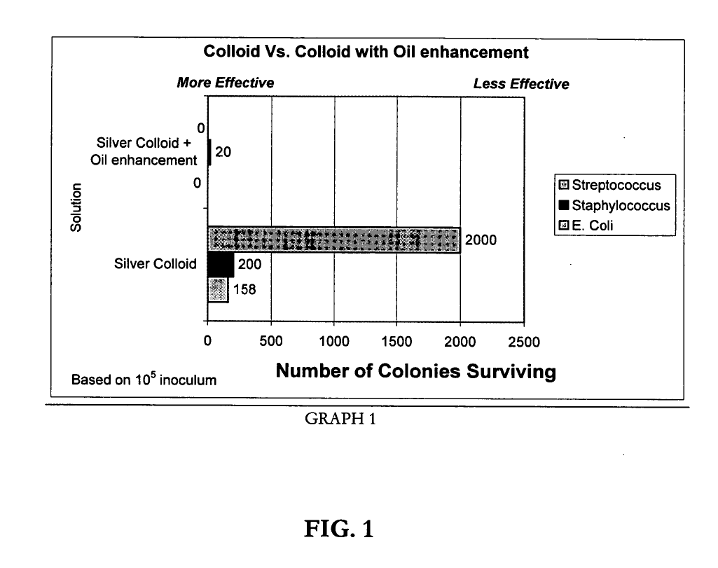

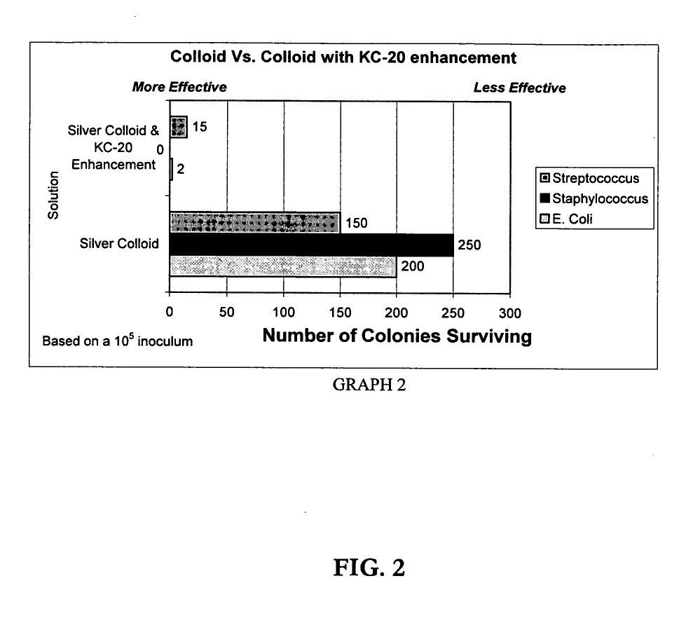

As shown in FIGS. 1 and 2, Graphs 1 and 2, the improvement provided by the enhancing agent varies depending on the particular organism. It can be seen however, that improvement range from a 10× improvement to a 2000× improvement. These are quite significant improvements.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. A method for enhancing the antimicrobial effectiveness of a silver colloid suspension against a desired bacteria by adding an agent to the colloid suspension which functions to modify the cell wall of the bacteria to tender the wall more penetrable to the silver in a colloidal suspension.

2. The method of claim 1, wherein the silver colloid suspension contains nanometer sized silver particles suspended in an aqueous carrier.

3. The method of claim 2, wherein the agent does not affect the silver particles ability to remain in suspension.

4. The method of claim 3, wherein the agent is peppermint oil.

5. The method of claim 3, wherein the agent is Polysorbate 20.

6. The method of claim 3, wherein the agent is a combination of both peppermint oil and Polysorbate 20.

7. The method of claim 1, wherein the agent is peppermint oil.

8. The method of claim 1, wherein the agent is Polysorbate 20.

9. The method of claim 1, wherein the agent is a combination of both peppermint oil and Polysorbate 20.

10. A method for enhancing the antimicrobial effectiveness of a silver colloid suspension against a desired bacteria by adding an agent to the colloid suspension which functions to reduce the surface tension of the peptides found in the cell walls of the bacteria and thus rendering them more penetrable to the silver in a colloidal suspension.

11. The method of claim 10, wherein the silver colloid suspension contains nanometer sized silver particles suspended in an aqueous carrier.

12. The method of claim 11, wherein the agent does not affect the silver particles ability to remain in suspension.

13. The method of claim 12, wherein the agent is peppermint oil.

14. The method of claim 12, wherein the agent is Polysorbate 20.

15. The method of claim 12, wherein the agent is a combination of both peppermint oil and Polysorbate 20.

16. The method of claim 10, wherein the agent is peppermint oil.

17. The method of claim 10, wherein the agent is Polysorbate 20.

18. The method of claim 10, wherein the agent is a combination of both peppermint oil and Polysorbate 20.
